(19) United States
(12) Patent Application Publication
(10) Pub. No.: US 2014/0041445 A1
(43) Pub. Date: Feb. 13, 2014

(54) APPARATUS FOR MEASURING DRYING RATE AND METHOD FOR MEASURING DRYING RATE USING THE SAME

(71) Applicant: SAMSUNG ELECTRO-MECHANICS CO., LTD., Suwon (KR)

(72) Inventors: Hee Sun Chun, Suwon (KR); Jae Choon Cho, Suwon (KR); Jong Yoon Jang, Suwon (KR); Dong Joo Shin, Suwon (KR); Sung Hyun Kim, Suwon (KR); Choon Keun Lee, Suwon (KR)

(21) Appl. No.: 13/937,595

(22) Filed: Jul. 9, 2013

(30) Foreign Application Priority Data
Aug. 9, 2012 (KR) 10-2012-0087384

(51) Int. Cl. G01N 33/32

(52) U.S. Cl. CPC G01N 33/32
USPC 73/150 R

(57) ABSTRACT

There are provided an apparatus for measuring a drying rate and a method for measuring a drying rate using the same in order to measure the drying rate of a substrate material for manufacturing an electronic apparatus, the apparatus for measuring a drying rate, including a support part having a substrate seated thereon, and a marking part disposed above the substrate while being vertically and horizontally movable, and forming a marking on the substrate while being in contact with the substrate.

### CROSS-REFERENCE TO RELATED APPLICATIONS

This application claims the priority of Korean Patent Application No. 10-2012-0087384 filed on Aug. 9, 2012, in the Korean Intellectual Property Office, the disclosure of which is incorporated herein by reference.

### BACKGROUND OF THE INVENTION

1. Field of the Invention

The present invention relates to an apparatus for measuring a drying rate and a method for measuring the drying rate using the same, and more particularly, to an apparatus for measuring a drying rate of a substrate material for manufacturing an electronic device and a method for measuring the drying rate using the same.

2. Description of the Related Art

In general, a printed circuit board (PCB) is provided with a wiring pattern by laminating a copper foil on a plate-shaped surface formed of an insulator such as resin, and performing processes such as pattern printing, etching, and the like, on the laminated copper foil, based on the design of a circuit.

The circuit board may be manufactured by a build-up process of laminating insulating films and a pattern formation process of forming the wiring pattern.

In addition, the insulating film used in the circuit board may be formed by a process of coating a raw material on a carrier layer using a molding device, a drying process of removing a solvent remaining in the insulating film, and the like.

Since a solvent content of the insulating film, that is, a drying rate of the insulating film, may cause several quality defects such as the occurrence of a non-coated portion in a product, a wrinkle defect due to high adhesive characteristics, and the like, through only a small change in the content thereof, the drying rate of the insulating film serves as a main factor in evaluating the quality of a final product.

In addition, the drying rate of the insulating film significantly affects a subsequent process and manufacturing efficiency, reliability, and the like of a package substrate, may be significantly affected by the drying rate of the insulating film.

However, in a case of the related art, since the drying rate of the insulating film may be determined by a worker using the naked eye or through tactile perception, a reference thereof may be very subjective. Therefore, a measuring error may be generated among many workers, and measurement reliability may be decreased.

### RELATED ART DOCUMENT

(Patent Document 1) Korean Patent Laid-Open Publication No. 1997-0049045

### SUMMARY OF THE INVENTION

An aspect of the present invention provides an apparatus for measuring a drying rate, capable of effectively and precisely measuring the drying rate of a substrate.

Another aspect of the present invention provides an apparatus for measuring a drying rate for automatically managing the drying rate by objectively digitizing the drying rate of a substrate.

Another aspect of the present invention provides a method for measuring a drying rate, capable of effectively and precisely measuring the drying rate of a substrate.

According to an aspect of the present invention, there is provided an apparatus for measuring a drying rate, the apparatus including: a support part having a substrate seated thereon; and a marking part disposed above the substrate while being vertically and horizontally movable, and forming a marking on the substrate while being in contact with the substrate.

The marking part may include: a roller part in contact with the substrate; and a driving part moving the roller part vertically and horizontally.

The roller part may include: a roller formed to have a cylindrical shape; and a frame coupled to the roller to thereby form a rotating shaft of the roller.

The roller may have a plurality of steps formed on an external surface thereof and the marking may be formed by the steps.

The roller may have the steps formed in a stepped manner such that outer diameters thereof decrease toward a center of the roller.

The roller may be formed to have the same interval therebetween.

The roller may include: a weight adjusting part formed to have a cylindrical pipe shape; and a step adjusting part formed of a plurality of rings having different sizes coupled to an external surface of the weight adjusting part.

The weight adjusting part may include a plurality of weight adjusting parts having the same shape and different weights and selectively coupled to the step adjusting part corresponding to characteristics of the substrate.

The roller may be coupled to the weight adjusting part such that the rings of the step adjusting part are spaced apart from each other by a predetermined distance.

The apparatus may further include a controlling part determining the drying rate of the substrate based on a shape of the marking formed on the substrate.

The apparatus may further include an imaging part imaging the shape of the marking formed on the substrate and sending image data regarding the imaged marking to the controlling part.

The controlling part may detect a depth of the marking from the shape of the marking and determine the drying rate of the substrate based on the depth of the marking.

According to another aspect of the present invention, there is provided a method for measuring a drying rate, the method including: seating a substrate on a support part; forming a marking while a roller contacts the substrate; and determining the drying rate of the substrate based on the marking.

The forming of the marking may include: seating the roller having a plurality of steps formed on an external surface thereof on the substrate; and rolling and moving the roller on the substrate.

The method may further include imaging the marking to obtain image data regarding the marking, after the forming of the marking.

In the determining of the drying rate, the image data of the marking may be compared to preset data and the drying rate may be measured.

In the determining of the drying rate, a depth of the marking may be detected by the image data regarding the marking, the depth of the marking may be compared to preset data, and the drying rate may be measured.

In the determining of the drying rate, the determination may be performed based on a remaining portion of the substrate except for a portion on which the roller is seated, in an overall region of the marking.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is a perspective view schematically showing an apparatus for measuring a drying rate according to an embodiment of the present invention;

FIG. 2 is a cross-sectional view of a roller part shown in FIG. 1;

FIG. 3 is a flowchart schematically showing a method for measuring a drying rate according to an embodiment of the present invention;

FIG. 4 is a side view showing a side of FIG. 1;

FIGS. 5A through 5C are views for describing processes of determining a drying rate of a substrate in the method for measuring the drying rate according to the embodiment of the present invention;

FIG. 6 is an exploded perspective view schematically showing a roller of an apparatus for measuring a drying rate according to another embodiment of the present invention; and

FIG. 7 is a cross-sectional view schematically showing a roller of an apparatus for measuring a drying rate according to another embodiment of the present invention.

### DETAILED DESCRIPTION OF THE EMBODIMENTS

Hereinafter, embodiments of the present invention will be described in detail with reference to the accompanying drawings.

The invention may, however, be embodied in many different forms and should not be construed as being limited to the embodiments set forth herein. Rather, these embodiments are provided so that this disclosure will be thorough and complete, and will fully convey the scope of the invention to those skilled in the art. In the drawings, the shapes and dimensions of elements may be exaggerated for clarity, and the same reference numerals will be used throughout to designate the same or like elements.

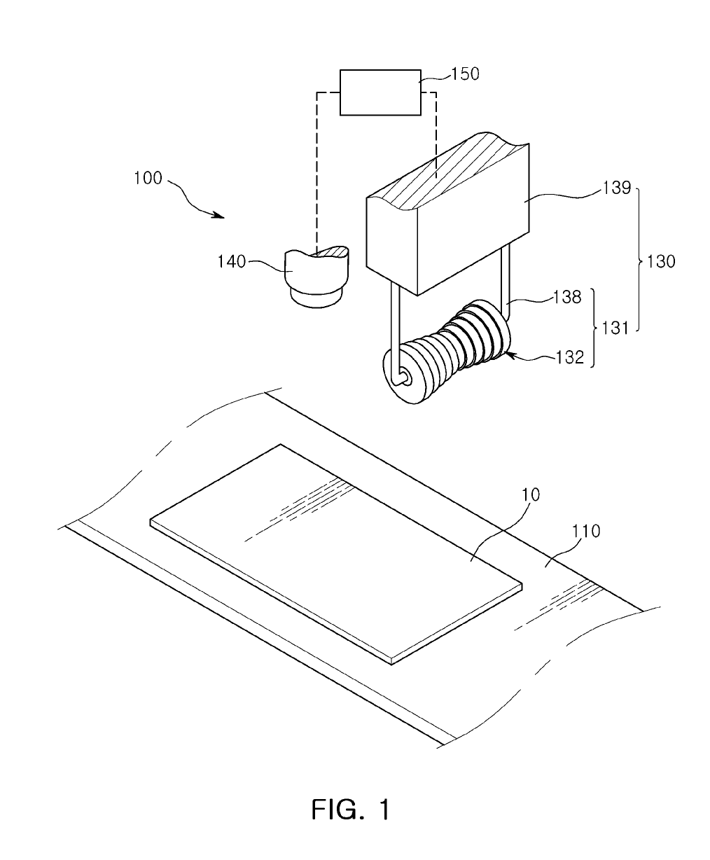

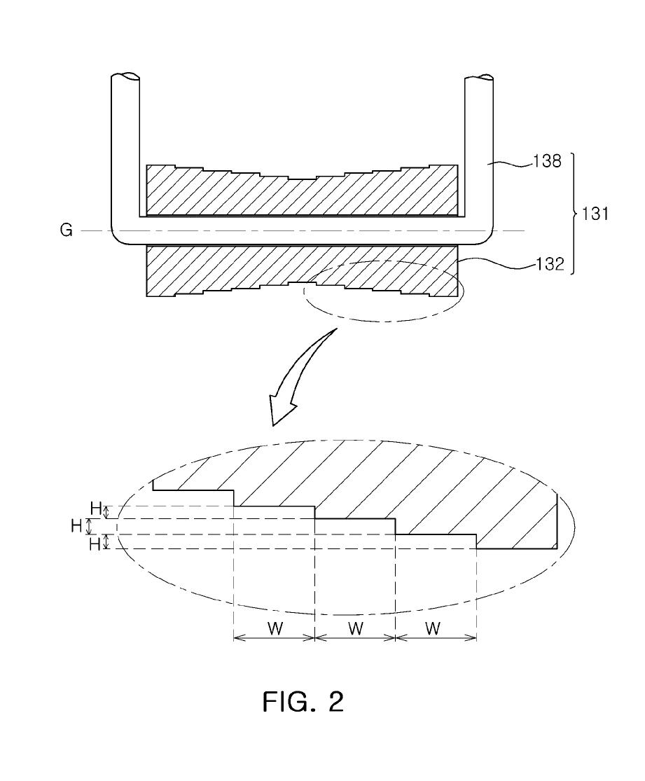

FIG. 1 is a perspective view schematically showing an apparatus for measuring a drying rate according to an embodiment of the present invention. FIG. 2 is a cross-sectional view of a roller part shown in FIG. 1.

Referring to FIGS. 1 and 2, an apparatus 100 for measuring a drying rate may include a support part 110, a marking part 130, an imaging part 140, and a controlling part 150.

The support part 110 may support a predetermined substrate 10. Specifically, the support part 110 may be formed in a form of a jig having a flat surface for supporting the substrate 10 such as an insulating substrate.

The marking part 130 may form a marking on the substrate 10 such as an insulating substrate disposed on the support part 110. To this end, the marking part 130 may include a roller part 131 and a driving part 139.

The roller part 131 may include a roller 132 and a frame 138.

The roller 132 may be formed to be elongated in a cylindrical pipe manner and may be rotated around a center shaft as a rotating shaft G.

The roller 132 may have a plurality of steps formed on an external surface thereof, as shown in FIG. 2. Here, the plurality of steps may be formed such that heights thereof increase toward both ends of the roller 132 having the cylinder shape and decrease toward the center of the roller 132 having the cylinder shape. That is, the roller 132 has the steps formed in a stepped manner such that outer diameters thereof are greatest at the both ends of the roller 132 and the outer diameters thereof are reduced toward the center of the roller 132.

The steps formed on the roller 132 may have the same interval H therebetween (that is, the same interval H therebetween in a vertical direction). For example, the interval between the steps in the vertical direction may be several nm to several μm. In addition, a width W between a step and an adjacent step may also be formed so as to have the same width.

The steps of the roller 132 are provided to form a marking on the substrate 10. That is, when the roller 132 is seated on the substrate 10, a marking caused by the steps is formed on the substrate 10 due to a weight of the roller 132.

Here, the marking may be formed in different manners according to a drying rate of the substrate 10. That is, when the substrate 10 is completely dried, even in a case in which the roller 132 is seated on the substrate 10, the marking may not be formed on the substrate 10. On the other hand, in the case in which an excess amount of solvent remains on the substrate 10, various stages of step markings (markings) may be formed on the substrate 10 by the steps of the roller 132.

Meanwhile, in order to form the marking more accurately, a material (for example, a color dye or the like) allowing the marking to be more clearly identified may be formed on the external surface of the roller 132, that is, a contacting surface in contact with the substrate 10.

The frame 138 may be inserted into the roller 132 and support the roller 132 such that the roller 132 is rotatably supported thereby. That is, the frame 138 may configure the rotating shaft G of the roller 132 and may be connected to the driving part 139 described below.

The driving part 139 may allow the roller 132 to be transferred. Specifically, the driving part 139 may be connected to the frame 138 and perform a vertical driving operation of seating the roller 132 on the substrate 10 or allowing the roller 132 to be spaced apart from the substrate 10, and a horizontal driving operation rolling the roller 132 on the substrate 10 when the roller 132 is seated on the substrate 10.

The imaging part 140 may be provided to detect the marking formed on the substrate 10 by the roller 132.

The imaging part 140 may include at least one camera. The camera images the marking formed on the substrate 10. Image data imaged by the camera may be sent to the controlling part 150 to be described below.

The controlling part 150 may analyze a shape of the marking, that is, the image data sent from the imaging part 140 and determine the drying rate of the substrate 10. In addition, in order to implement more precise measurement, the imaging part 140 is controlled, such that a position of the camera may also be changed.

Meanwhile, in the case in which the worker directly identifies the marking with the naked eye, the imaging part 140 and the controlling part 150 may be omitted.

In addition, the controlling part 150 may be connected to the driving part 139 of the marking part 130 to thereby control the driving operations of the driving part 139. That is, the controlling part 150 may sequentially and repeatedly control an operation of forming the marking by driving the driving part 139 and imaging the marking by driving the imaging part 140. However, in the case in which the driving part 139 includes a separate controlling unit, the control of the driving part 139 may be omitted.

Continuously, a process for measuring the drying rate of the apparatus 100 for measuring a drying rate according to the embodiment of the present invention described above will be described in detail. Here, descriptions overlapped with those of the apparatus 100 for measuring a drying rate described above may be omitted or simplified.

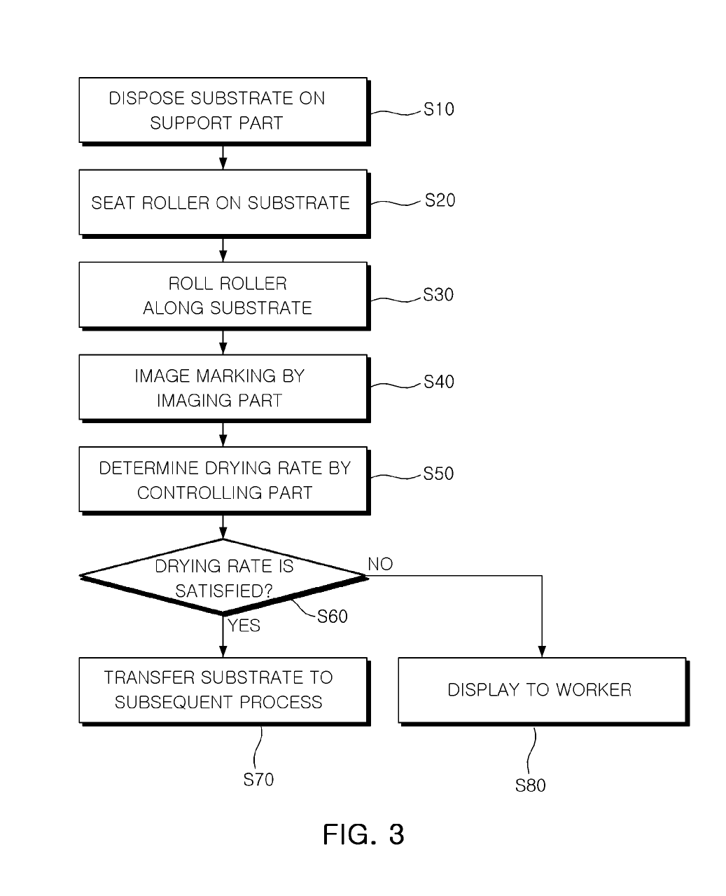

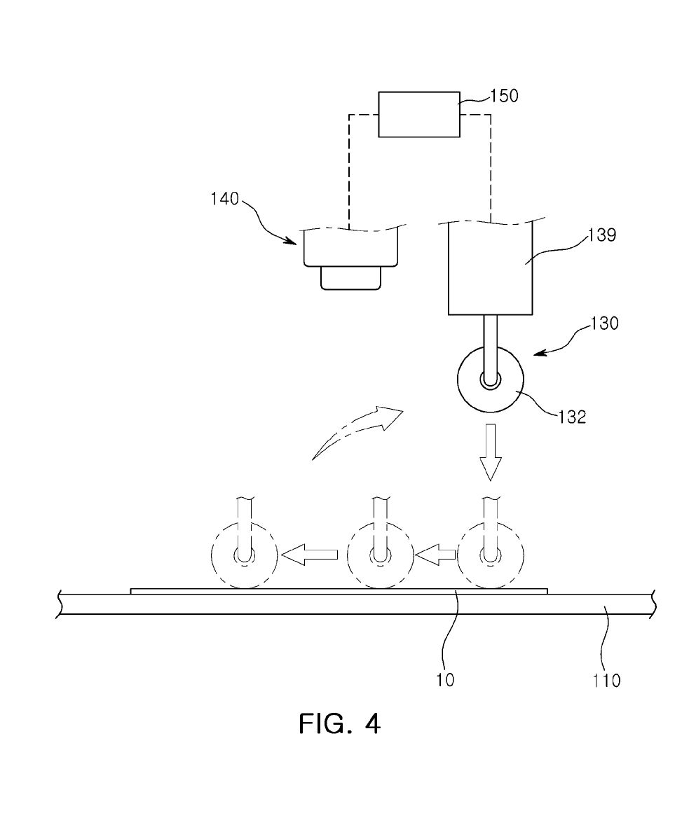

FIG. 3 is a flow chart schematically showing a method for measuring a drying rate according to an embodiment of the present invention. FIG. 4 is a side view showing a side of FIG. 1.

Referring to FIGS. 3 and 4, first, the substrate 10 which has been subjected to a drying process is disposed on the support part 110 (S10). Here, the substrate 10 may be an insulating film used in manufacturing a printed circuit board (PCB) and the drying process may be a process of drying the insulating film to remove a solvent in the insulating film by 90% or more.

In addition, the substrate 10 may be a part of the insulating film to be measured. For example, the substrate 10 may be a part (hereinafter, referred to as a sample substrate) of the insulating film formed by cutting one region of the insulating film. As described above, in the case in which a drying rate of the entire insulating film is measured by only collecting a part of the insulating film, the drying rate of the insulating film may be measured by measuring respective drying rates of various sample substrates collected in different regions of the insulating film and determining whether or not the respective drying rates mostly or entirely satisfy a preset drying rate.

In addition, the drying rate of the insulating film may be measured by determining whether or not an average value of the respective drying rates of the sample substrates 10 satisfies a preset average value.

Meanwhile, the support part 110 may include an apparatus (not shown) for absorbing the substrate 10 with vacuum pressure such that the substrate 10 may be firmly fixed to the support part 110.

Next, the marking is formed on the substrate 10. In the forming of the marking, a process of seating the roller 132 on the substrate 10 is first performed (S20).

The driving part 139 may allow the roller 132 to be transferred and seat the roller 132 in a position adjacent to one side of the substrate 10. Therefore, the external surface of the roller 132 may be in contact with one surface of the substrate 10, such that the substrate 10 is pressurized with pressure corresponding to the weight of the roller 132.

In this process, since the roller 132 falls toward the substrate 10 at a predetermined speed from the above of the substrate 10 and is seated on the substrate 10, the substrate 10 is pressurized by the weight of the roller 132 as well as impacts generated by gravity during the falling of the roller. Therefore, in the position of the substrate 10 in which the roller 132 is firstly seated, the substrate 10 is pressurized by greater force in addition to the weight of the roller 132.

Due to this fact, since the marking formed in the position on which the roller 132 is seated includes an error, it is difficult to precisely measure the drying rate of the substrate 10.

In order to solve the defect, in the method for measuring the drying rate according to the embodiment of the present invention, a method of rolling and moving the roller 132 on the substrate 10 for a predetermined distance.

That is, when the roller 132 is seated on the substrate 10, a process of rolling the roller 132 is performed (S30). The driving part 139 linearly moves the frame 138 to the other side of the substrate 10. Therefore, the roller 132 coupled to the frame 138 rolls in a state of pressurizing the substrate 10 using its weight and is moved to the other side of the substrate 10.

In this case, in order to form the marking only by using the weight of the roller 132, a predetermined space may be formed between the frame 138 inserted into the roller 132 and an inner peripheral surface of the roller 132. Therefore, when the roller 132 is rolled, no force in addition to the weight of the roller 132 is reflected on the substrate. Through the processes, the marking is formed on the substrate 10 by the weight of the roller 132 and the steps of the external surface of the roller 132. In this case, the formed marking may be linearly extended in a movement path of the roller 132.

Meanwhile, in this process, the driving part 139 may be moved horizontally so that the roller 132 rolls about once (a single rotation thereof). Therefore, the linear marking formed by the roller 132 may be formed to be equal to the circumference of the roller 132. However, the present invention is not limited thereto.

Next, a process of removing the roller 132 from the substrate 10 is performed. The driving part 139 moves the roller 132 toward above the substrate 10, separates the roller 132 and the substrate 10 from each other, and disposes the roller 132 in an initial position (that is, above one side of the substrate).

Next, the drying rate is determined.

First, the imaging part 140 images a marking-formed surface of the substrate (S40). In addition, the imaged image data is sent to the controlling part 150. Therefore, the controlling part 150 determines whether or not the drying rate of the substrate 10 satisfies the preset drying rate (S50).

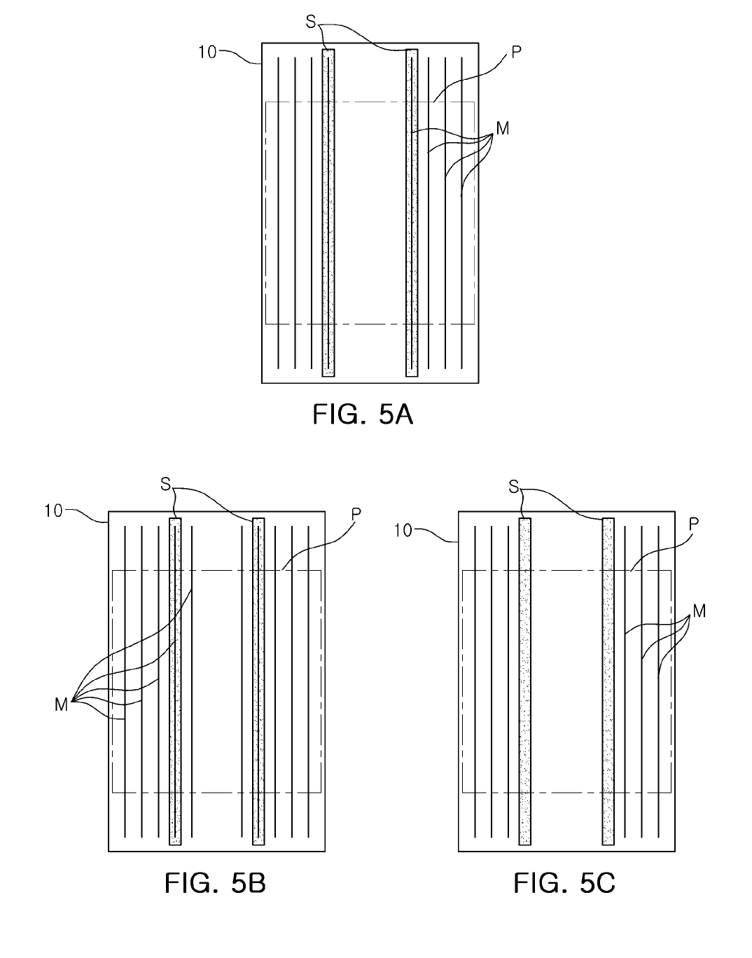

FIGS. 5A through 5C are views for describing processes of determining a drying rate of a substrate in the method for measuring the drying rate according to the embodiment of the present invention and show different cases of the image data imaged by the imaging part 140.

Referring to FIGS. 5A through 5C, the controlling part 150 analyzes the image data, compares the marking-formed region of the substrate 10 with preset data, and measures the drying rate.

Here, the preset data may use various forms of data and may be a sign indicating specific lines S (hereinafter, referred to as a reference line) overlapped with the image data as shown in FIGS. 5A through 5C. In this case, the controlling part 150 may determine the drying rate by overlapping lines M formed on the marking with the reference lines S to determine whether or not the lines M formed on the marking correspond to the reference lines S.

For example, as shown in FIG. 5A, in the case in which the lines M formed on the marking of the substrate 10 are only formed up to the reference lines S, the controlling part 150 may determine that the drying rate of the substrate 10 satisfies the preset drying rate (S60). In this case, the controlling part 150 may transfer the substrate 10 to a place in which a subsequent process is performed (S70).

On the other hand, as shown in FIG. 5B, in the case in which the lines M formed on the marking are also formed inside the reference lines S, the controlling part 150 may determine that the drying rate of the substrate 10 is lower than the preset drying rate. That is, the controlling part 150 may determine that the drying process of the substrate 10 is not performed sufficiently.

In addition, as shown in FIG. 5C, in the case in which the lines M formed on the marking are not formed up to the reference lines S, the controlling part 150 may determine that the drying rate of the substrate 10 is higher than the preset drying rate. That is, the controlling part 150 may determine that the drying process of the substrate 10 is performed excessively.

In the case in which the controlling part 150 determines that the drying rate of the substrate 10 satisfies the reference drying rate (S60), the controlling part 150 may display the determined result to a worker or may separately classify the substrate 10 as an abnormal and defective substrate 10 (S80).

Meanwhile, since a position of the substrate in which the roller 132 is initially seated has force (that is, impact force) applied thereto, in addition to the weight of the roller 132, more lines may be formed in the marking. Therefore, the controlling part 150 may not consider the position of the substrate in which the roller 132 is firstly positioned in the overall shape of the marking.

That is, the controlling part 150 may perform the determination with respect to the remaining region (P of FIGS. 5A through 5C) except for the position of the substrate 10 in which the roller 132 is first seated (a portion on which the marking is started), in the overall region of the marking.

Meanwhile, the method for measuring the drying rate according to the embodiment of the present invention is not limited to the configuration using the reference lines and various methods may be used as needed. For example, the controlling part 150 may determine the drying rate based on a depth of the marking formed on the substrate 10.

As described above, the roller 132 according to the present embodiment may be configured such that all of the steps have the same interval. Therefore, when one step has an interval of 1 μm and four step markings M are formed on the substrate 10 as shown in FIG. 5A, it may be appreciated that a maximum depth of the marking is 3 μm to 4 μm. That is, the depth of the marking may be detected based on the number of the step markings M and the drying rate may be determined based on the depth.

In this case, drying rate data according to various depths of the markings may be preset in the controlling part 150. Here, the drying data may be data formed by digitizing a material or thickness of the substrate 10, the weight of the roller 132, the depth of the marking at the corresponding drying rate, or the like.

As set forth above, the apparatus 100 for measuring the drying rate and the method for measuring the drying rate using the same may include the marking part 130 forming the marking on the substrate 10, the imaging part 140 imaging the image for the marking-formed region formed on the substrate 10, and a controlling part 150 determining the drying rate of the substrate 10 according to the distribution degree of the marking-formed region of the substrate 10 imaged by the imaging part 140.

Therefore, the drying rate of the substrate 10 may be objectively measured and the process for measuring the drying rate of the substrate 10 may be objectified, refined, and automated by controlling the subsequent process of the substrate 10 according to whether or not the drying rate of the substrate 10 satisfies the preset drying rate.

In addition, since the marking may be formed on the substrate 10 only by the process of rolling the roller 132 on the substrate 10, the marking may be very easily formed.

In addition, since the marking is formed by the weight of the roller and the steps formed on the roller, the depth of the marking may be easily detected to thereby measure the drying rate.

Meanwhile, the apparatus for measuring the drying rate according to the embodiment of the present invention is not limited to the above described embodiments, but may be configured in various forms, as needed.

In general, the substrate (for example, the insulating film) may have different degrees (the depth or the like) to which the markings are formed, according to a material, a thickness, a drying rate, and the like thereof. Therefore, the following embodiment provides an apparatus capable of measuring a drying rate while the worker changes the weight of the roller according to a state, a type, and the like of the substrate.

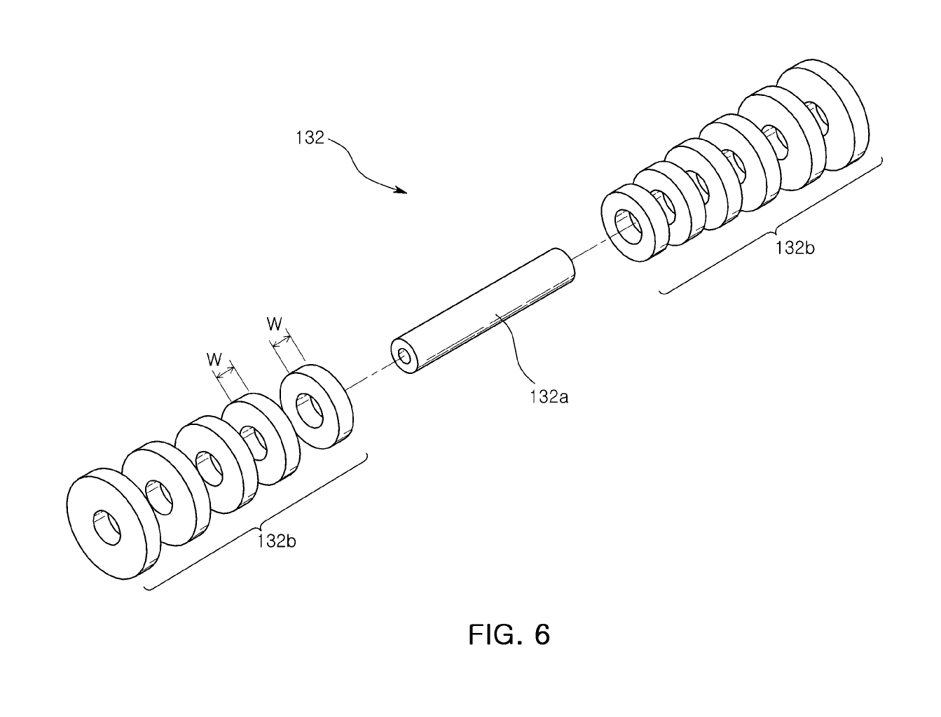

FIG. 6 is an exploded perspective view schematically showing a roller of an apparatus for measuring a drying rate according to another embodiment of the present invention.

Referring to FIG. 6, the roller 132 according to the present embodiment is not integrally formed and is formed by coupling a weight adjusting part 132a to step adjusting parts 132b.

The weight adjusting part 132a is formed to have a cylindrical pipe shape, while having an empty space therein. Particularly, the weight adjusting part 132a according to the present embodiment may include a plurality of weight adjusting parts 132a having the same shape, but having different weights.

Therefore, the roller 132 according to the present embodiment may be configured by selecting the weight adjusting part 132a corresponding to a required weight and coupling the step adjusting parts 132b to be described below to the weight adjusting part 132a.

The above-mentioned configuration is a derived so as to change the weight of the roller 132, as needed. That is, the worker may configure the roller 132 by coupling the weight adjusting part 132a to the step adjusting parts 132b to form an appropriate weight thereof as needed. Therefore, the apparatus 100 for measuring the drying rate according to the embodiment of the present invention may also be easily applied to various substrates 10 having different materials or thicknesses.

The step adjusting parts 132b may respectively include a plurality of rings having different sizes.

Here, the plurality of rings of the step adjusting part 132b have outer diameters decreased by a predetermined amount and all of inner holes are formed to have sizes corresponding to outer diameters of the weight adjusting part 132a. In addition, the respective rings may be formed to have the same width W.

The step adjusting parts 132b may be coupled to an external surface of the weight adjusting part 132a to thereby complete the roller 132. In this case, the step adjusting parts 132b may be coupled so that the rings having the largest outer diameter are disposed at both ends of the weight adjusting part 132a and the rings having small outer diameters are disposed toward the center of the weight adjusting part 132a. Therefore, the overall exterior of the roller 132 according to the present embodiment may be formed to have the same shape as that of the roller (132 of FIG. 1) of the above described embodiment.

Meanwhile, the present embodiment exemplifies a case in which the all of the rings of the step adjusting part 132b are formed to have the same width W. However, the present invention is not limited thereto. For example, various applications may be made as needed. For example, the rings may have a wider or narrower width towards the center of the roller 132.

In addition, the step adjusting part 132b according to the present embodiment may include various rings based on the sizes of the outer diameters to thereby configure rollers having various sizes.

For example, two rollers (hereinafter, referred to a first roller and a second roller) assembled, may be respectively configured so as to have the same step between the respective rings coupled to the weight adjusting part 132a, but have a difference in the overall steps of the roller.

In this case, since the first and second rollers have outer diameters different from each other, the weights of the respective step adjusting parts 132b may be different. However, as described above, since the weight adjusting part 132a according to the present embodiment includes the plurality of weight adjusting parts 132a having the same shape, but having the different weights, the first and the second rollers may be configured so as to have the same weight by adjusting the weights of the weight adjusting parts 132a.

As such, since the roller 132 according to the present embodiment may adjust the weight thereof only using the weight adjusting part 132a, even when the assembled roller 132 has different outer diameters, the roller to which the same number of rings are coupled may constantly have the same weight.

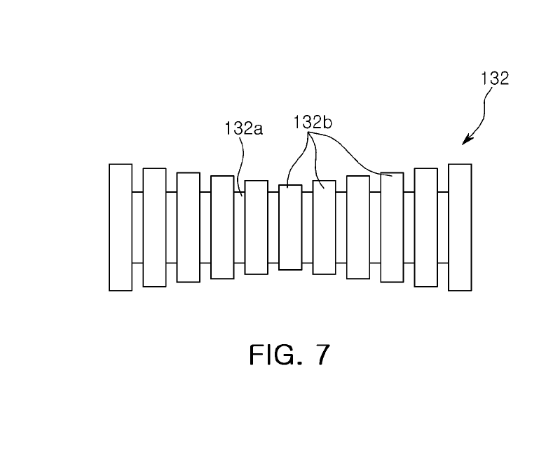

FIG. 7 is a cross-sectional view schematically showing a roller of an apparatus for measuring a drying rate according to another embodiment of the present invention and shows an example modifying the roller shown in FIG. 6.

Referring to FIG. 7, in the roller 132 according to the present embodiment, rings of the step adjusting part 132b may be coupled to the weight adjusting part 132a while being spaced apart from each other by a predetermined interval, rather than being disposed to continuously contact each other.

In this case, since the roller 132 may be configured using a relatively small number of rings, manufacturing costs may be decreased.

Meanwhile, although the present embodiment describes the case in which the roller 132 is configured by coupling the weight adjusting part 132a to the step adjusting part 132b, as an example, various applications may be made. For example, the weight adjusting part 132a and the step adjusting part 132b may be integrally formed in the manufacturing process as in the roller 132 shown in FIG. 1.

The apparatus for measuring the drying rate and the method for measuring the drying rate according to the present invention as described above are not limited to the above-mentioned embodiments, and those skilled in the art will appreciate that various modifications, additions and substitutions are possible, without departing from the scope and spirit of the invention.

For example, although the above described embodiments describe the case in which the roller is configured such that the steps thereof decrease toward the center thereof, as an example, the present invention is not necessarily limited thereto. For example, the roller may be configured such that the steps thereof increase toward the center of the roller.

In addition, although the above described embodiments describe the apparatus and the method for measuring the drying rate of the substrate, that is, the insulating film, as an example, the present invention may be easily applied to various apparatuses or fields of the application in which a flat plate is formed to allow for the measurement of the drying rate thereof, rather than using the substrate.

As set forth above, the apparatus for measuring the drying rate may include the marking part forming the marking on the substrate, the imaging part imaging the image for the marking-formed region formed on the substrate, and the controlling part determining the drying rate of the substrate according to the distribution degree of the marking-formed region of the substrate imaged by the imaging part.

Therefore, the drying rate of the substrate can be objectively measured and the process for measuring the drying rate of the substrate can be objectified, refined, and automated by controlling the subsequent process of the substrate according to whether or not the drying rate of the substrate satisfies the preset drying rate.

In addition, since the method for measuring the drying rate according to the embodiment of the present invention can form the marking on the substrate only by the process of rolling the roller on the substrate, the marking can be very easily formed.

In addition, since the marking is formed by the weight of the roller and the step formed on the roller, the depth of the marking can be easily detected to thereby easily measure the drying rate.

While the present invention has been shown and described in connection with the embodiments, it will be apparent to those skilled in the art that modifications and variations can be made without departing from the spirit and scope of the invention as defined by the appended claims.

What is claimed is:

1. An apparatus for measuring a drying rate, the apparatus comprising:
a support part having a substrate seated thereon; and
a marking part disposed above the substrate while being vertically and horizontally movable, and forming a marking on the substrate while being in contact with the substrate.

2. The apparatus of claim 1, wherein the marking part includes:
a roller part in contact with the substrate; and
a driving part moving the roller part vertically and horizontally.

3. The apparatus of claim 2, wherein the roller part includes:
a roller formed to have a cylindrical shape; and
a frame coupled to the roller to thereby form a rotating shaft of the roller.

4. The apparatus of claim 3, wherein the roller has a plurality of steps formed on an external surface thereof and the marking is formed by the steps.

5. The apparatus of claim 4, wherein the roller has the steps formed in a stepped manner such that outer diameters thereof decrease toward a center of the roller.

6. The apparatus of claim 5, wherein the roller has the steps formed to have the same interval therebetween.

7. The apparatus of claim 2, wherein the roller includes:
a weight adjusting part formed to have a cylindrical pipe shape; and
a step adjusting part formed of a plurality of rings having different sizes coupled to an external surface of the weight adjusting part.

8. The apparatus of claim 7, wherein the weight adjusting part includes a plurality of weight adjusting parts having the same shape and different weights and selectively coupled to the step adjusting part corresponding to characteristics of the substrate.

9. The apparatus of claim 7, wherein the roller is coupled to the weight adjusting part such that the rings of the step adjusting part are spaced apart from each other by a predetermined distance.

10. The apparatus of claim 1, further comprising a controlling part determining the drying rate of the substrate based on a shape of the marking formed on the substrate.

11. The apparatus of claim 10, further comprising an imaging part imaging the shape of the marking formed on the substrate and sending image data regarding the imaged marking to the controlling part.

12. The apparatus of claim 10, wherein the controlling part detects a depth of the marking from the shape of the marking and determines the drying rate of the substrate based on the depth of the marking.

13. A method for measuring a drying rate, the method comprising:
seating a substrate on a support part;
forming a marking while a roller contacts the substrate; and
determining the drying rate of the substrate based on the marking.

14. The method of claim 13, wherein the forming of the marking includes:
seating the roller having a plurality of steps formed on an external surface thereof on the substrate; and
rolling and moving the roller on the substrate.

15. The method of claim 14, further comprising imaging the marking to obtain image data regarding the marking, after the forming of the marking.

16. The method of claim 15, wherein in the determining of the drying rate, the image data regarding the marking is compared to preset data and the drying rate is measured.

17. The method of claim 15, wherein in the determining of the drying rate, a depth of the marking is detected by the image data regarding the marking, the depth of the marking is compared to preset data, and the drying rate is measured.

18. The method of claim 14, wherein in the determining of the drying rate, the determination is performed with respect to a remaining portion of the substrate except for a portion on which the roller is seated, in an overall region of the marking.
